(19) United States
(12) Patent Application Publication
(10) Pub. No.: US 2019/0183675 A1
(43) Pub. Date: Jun. 20, 2019

(54) EYE DROP APPLICATOR AND METHOD

(71) Applicant: Edwin Ryan, St. Paul, MN (US)

(72) Inventor: Edwin Ryan, St. Paul, MN (US)

(21) Appl. No.: 16/318,935

(22) PCT Filed: Jul. 20, 2017
(86) PCT No.: PCT/US17/43144

§ 371 Date: Jan. 18, 2019

Related U.S. Application Data
(60) Provisional application No. 62/364,691, filed on Jul. 20, 2016.

(51) Int. Cl. A61F 9/00

(52) U.S. Cl. CPC A61F 9/0026

(57) ABSTRACT

Devices and methods to apply eye drops are shown. Example devices include carrier strips with an amount of eye medication included on or within at least one end. In selected methods, a patient touches an end including an amount of medication to their eye, and an amount of the medication is transferred from the carrier to their eye.

### CLAIM OF PRIORITY

This application claims the benefit of priority to U.S. Provisional Patent Application Ser. No. 62/364,691, filed on Jul. 20, 2016, which is hereby incorporated by reference herein in its entirety.

### BACKGROUND

Eye drops can be difficult to administer for members of the general public. The medication is often expensive, and a typical eye drop has a volume of 50 microliters which is more than double what is needed for therapeutic purposes. Also, a large percentage of the medication is often wasted when an user improperly applies eye drops using a dropper and misses their eye. Additionally, using a dropper may result in accidental touching of a user's eye with the tip of the dropper. This may be an undesirable source of possible infection or trauma to the eye.

Improved application devices for eye drop medication and methods are desired that address these and other problems.

### BRIEF DESCRIPTION OF THE DRAWINGS

FIG. 1A shows a strip according to an embodiment of the invention.

FIG. 1B shows another strip according to an embodiment of the invention.

FIG. 2 shows an application of an eye drop according to an embodiment of the invention.

FIG. 3 shows a dispenser according to an embodiment of the invention.

FIG. 4 shows a method of applying an eye drop according to an embodiment of the invention.

FIG. 5 shows another method of applying an eye drop according to an embodiment of the invention.

FIG. 6 shows another method of applying an eye drop according to an embodiment of the invention.

FIG. 7 shows another application of an eye drop according to an embodiment of the invention.

FIG. 8 shows a device for application of an eye drop according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

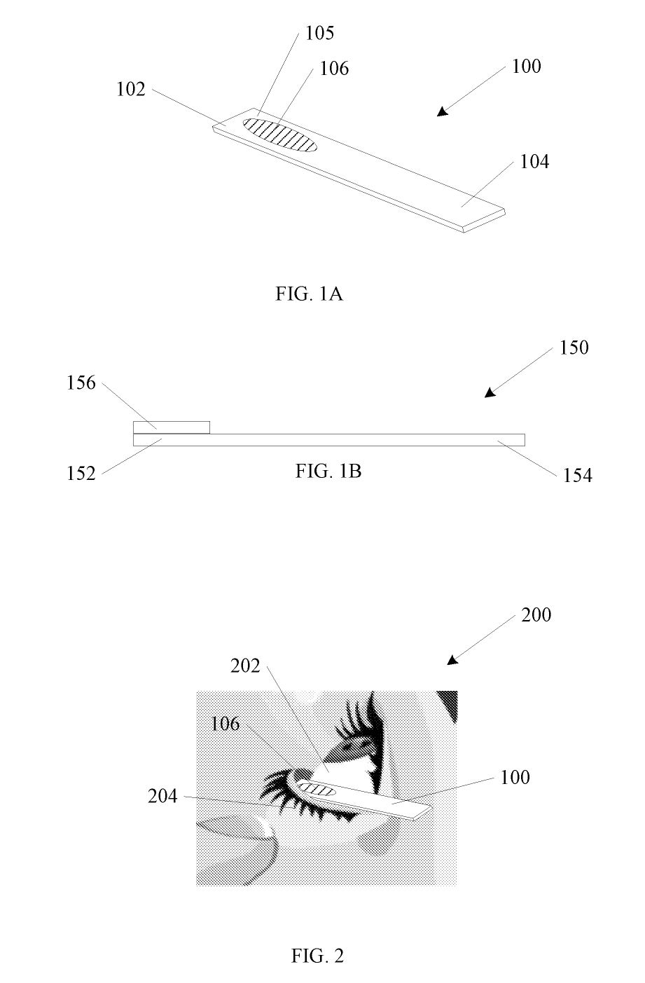

FIG. 1A shows a strip 100 according to one example. The strip 100 includes a first end 102 and a second end 104. An amount of eye medication 106 is located in a region 105 at the first end 102 of the strip 100. In one example, the strip 100 is a section of material that has been torn from a larger roll of material.

In one example, the strip 100 is formed from a material that at least partially absorbs an eye drop that contains the amount of eye medication 106. When the first end 102 of the strip 100 is placed in contact with an eye, some portion of the eye drop is transferred from the first end 102 of the strip 100 to the eye.

In one example, the strip 100 is formed from a first material, while the region 105 is formed from a second material with different properties from the first material. In one example, the region 105 is formed from a material that is more hydrophilic than the material of the surrounding strip 100. In one example, the region 105 is formed from a material that is more hydrophobic than the material of the surrounding strip 100.

In one example the strip 100 is formed from paper. In one example the dimensions of the paper strip 100 are such that the strip 100 is stiff enough to accurately place the medication 106 in the eye, and still flexible enough to bend when in contact with the eye, and not cause any physical damage. Paper or similar materials have an advantage of having a level of absorbency. Other materials with similar properties apart from paper are also within the scope of examples of the invention.

In one example, tearing of a paper strip 100 or strip 100 of a similar fibrous material, creates fibrils at the tear location that are beneficial. In one example, the fibrils aid in absorption of the medication 106. In one example the fibrils further provide a soft end that is comfortable for the patient to use when touching their eye. In other hydrophobic examples, the presence of some fibrils provides a level of adhesion of a droplet of medication sufficient to hold the droplet in place during application, while the generally hydrophobic nature of the material (paper, fabric, or the like) allows the droplet to bead up, and be more easily released when applied to the eye. In one example, the fibrils provide sufficient force to hold a droplet in place with the strip inverted, but are still hydrophobic enough to easily release the droplet when applied to the eye.

In one example, the region 105 includes other differences from the surrounding strip 100 apart from material choice. In one example, the region 105 includes a texture or geometry that modifies adhesion properties of the amount of eye medication 106. In one example, adhesion is increased to promote stability of the amount of eye medication 106. In one example, adhesion is decreased to promote release of the amount of eye medication 106 once it touches an eye.

FIG. 2 shows application of the strip 100 to a space between a lower eyelid 204 and an eye 202. The amount of eye medication 106 is shown in contact with the eye 202. Once in contact, an amount of the eye medication 106 will transfer to the eye 202. Because the eye medication is adherent to the strip, the problems associated with missing an eye with a conventional eye drop are eliminated. If a patient misses their eye 202 on a first attempt, the eye medication 106 will not spill onto the patient's cheek. A second attempt to apply the eye medication 106 can be made without wasting an eye drop. Further, because the strip 100 can be disposable, problems associated with bacterial contamination on a dropper end of conventional eye drop bottles is eliminated.

In one example, a portion, or all of the strip 100 is capable of indicating when an eye drop has been applied to the strip 100. In one example only the region 105 is capable of indicating when an eye drop has been applied to the region 105. In one example, a chemical is added to change color or otherwise indicate a presence of an applied eye drop to the first end. In other examples, the entire strip 100 includes an amount of eye medication. In one example, a patient applies the eye drop themselves before touching their eye. In another example, the strip 100 is pre-fabricated with the eye medication already in the strip.

One advantage of having the patient applying the eye drop to the strip themselves is that current eye drops do not need to be modified to use this application procedure. A patient merely adds the desired amount of eye drop medication to the strip 100 instead of directly to their eye.

In one example, at least a portion of the strip 100 is dissolvable. In one example only the region 105, or a distal end of the strip 100 is dissolvable. In one example, an entire strip 100 is dissolvable. In a dissolvable example, the patient touches their eye, and at least a portion of the strip 100 dissolves and transmits at least a portion of eye medication included within the dissolved portion of the strip 100.

FIG. 1B shows an example of a strip 150, including a first end 152 and a second end 154 with a dissolvable portion 156 coupled to the first end 152. In one example, the dissolvable portion 156 is hydrophilic, and is dimensioned to only absorb a medical necessary amount of medication. In one example, the dissolvable portion 156 includes a pre-formed medical necessary amount of medication. In the example of FIG. 1B, because the dissolvable portion 156 is laminated or otherwise attached to the strip 150, it is easier to control a size of the dissolvable portion 156, and as a result, the dosage of absorbed medication.

Similar to the example above, in one example, the patient applies the eye drop themselves to the dissolvable dispenser strip before touching their eye. In another example, the strip 100 is pre-fabricated with the eye medication already in the strip.

In one example, a strip 100 as shown may be incorporated into a roll. In use, a patient may pull out a useful length of strip 100 and tear off a portion for use. In one example, weakened seams such as perforations, may be used to indicate a desired length of strip 100 from a roll. In other examples, the patient may tear off any desired length of strip 100, without any pre-selected length. In one example, a roll of strip material is included in a dispenser with an edge that facilitates tearing off a length in a strip 100.

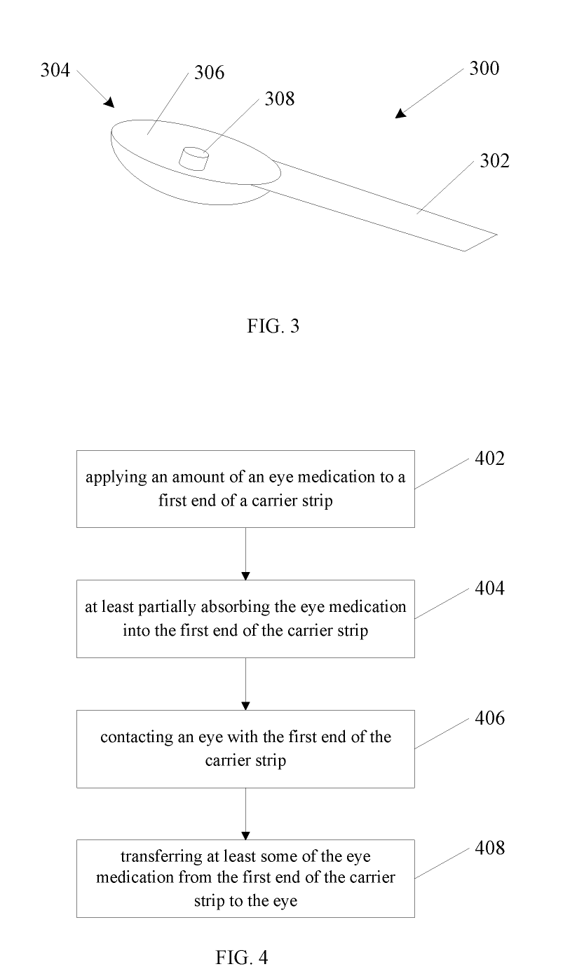

FIG. 3 shows a dispenser 300 according to one example. In the example of FIG. 3, a handle 302 is included with a drop holder 304 located at one end. A protrusion 308 may be included with the drop holder 304. In one example, the protrusion 308 aids in retention of an eye drop, using surface tension within the liquid eye drop to hold the eye drop in place before application to an eye. In the example shown, the drop holder 304 further includes a scoop 306. In one example, the combination of the scoop 306 and the protrusion 308 further enhances drop retention.

Although a scoop shape with a cylindrical protrusion is shown in the example of FIG. 3, the invention is not so limited. Other geometries apart from scoops and other protrusions apart from cylinders may be used to enhance surface tension in an eye drop.

Similar to examples discussed above, it can be advantageous to use pre-existing eye drops in combination with a dispenser, such as dispenser 300. Current eye drops do not need to be modified to use this application procedure. A patient merely adds the desired amount of eye drop medication to the dispenser 300 instead of directly to their eye.

Another advantage of example configurations shown includes the ability to reduce a drop size. Using examples described in the present disclosure, an amount of medication 106 present in each drop can be reduced to only a medically necessary amount with less waste. This reduces cost for the patient, but extending the lifetime of a bottle of medication 106.

In one example, an absorption property of a strip 100 can be chosen to only absorb a medically necessary amount of medication 106. In one example, a material choice and geometry for the region 105 can be chosen to only hold a drop sized for a medically necessary amount of medication 106.

FIG. 4 shows a flow diagram of one example method of application. In operation 402, an amount of an eye medication is applied to a first end of a carrier strip. In operation 404, the eye medication is at least partially absorbed into the first end of the carrier strip. In operation 406, an eye is contacted with the first end of the carrier strip, and in operation 408, at least some of the eye medication is transferred from the first end of the carrier strip to the eye.

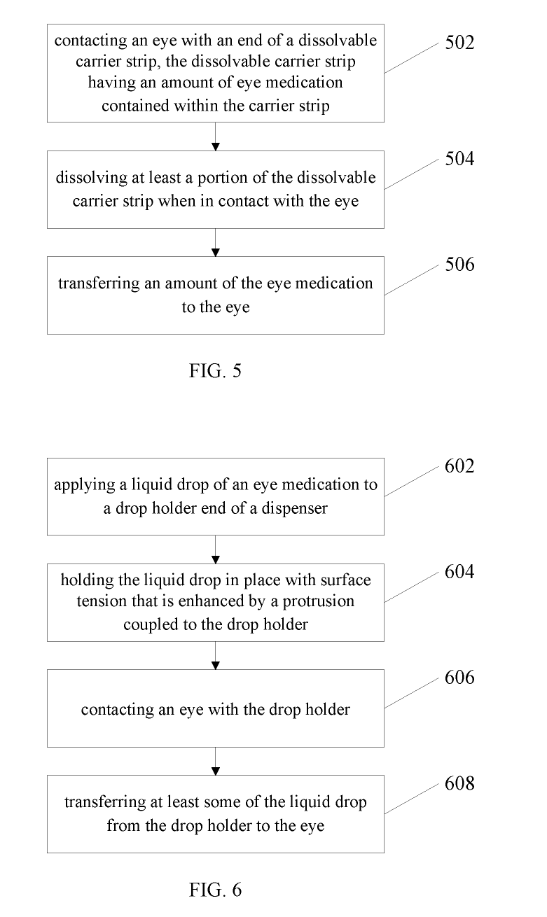

FIG. 5 shows another flow diagram of one example method of application. In operation 502, an eye is contacted with an end of a dissolvable carrier strip. The dissolvable carrier strip includes an amount of eye medication contained within the carrier strip. In operation 504, at least a portion of the dissolvable carrier strip is dissolved when in contact with the eye. In operation 506, an amount of the eye medication is transferred to the eye.

FIG. 6 shows another flow diagram of one example method of application. In operation 602, a liquid drop of an eye medication is applied to a drop holder end of a dispenser. In operation 604, the liquid drop is held in place with surface tension that is enhanced by a protrusion coupled to the drop holder. In operation 606, an eye is contacted with the drop holder. In operation 608, at least some of the liquid drop is transferred from the drop holder to the eye.

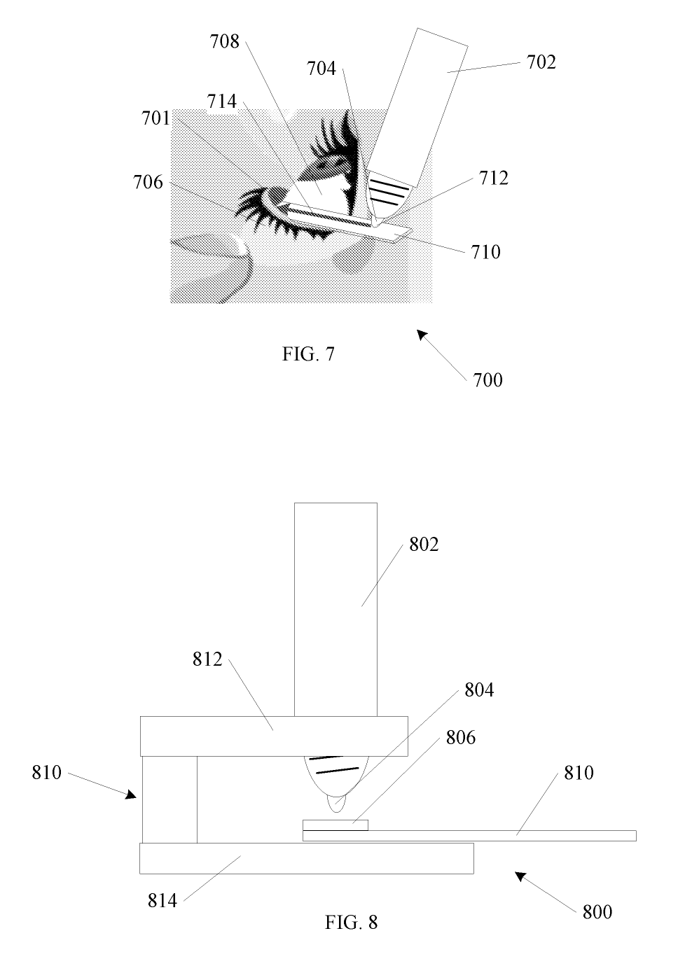

FIG. 7 shows a method of applying an amount of medication using another combination of devices and methods according to an embodiment of the invention. In the example of FIG. 7, a strip 710 is applied to a space 701 between a lower eyelid 706 and an eye 708. In the example described in FIG. 7, a standard eye drop bottle 702 is used to apply an amount of eye medication 712 to a location 704 on the strip 710 that is remote from the eye 708. In one example, the strip 710 is hydrophilic, and the amount of eye medication 712 is at least partially absorbed into the strip 710, where it travels by capillary action along direction 714 to the space 701 between the lower eyelid 706 and the eye 708.

In another example, the strip 710 is hydrophobic, and the amount of eye medication 712 is channeled on a surface of the strip 710 using a feature such as a ridge, or a recess, or a difference in material. In this example, the amount of eye medication 712 likewise travels along direction 714 to the space 701 between the lower eyelid 706 and the eye 708.

In both examples, the user is more comfortable using the method because the standard eye drop bottle 702 is not positioned directly over the eye 708. In one example, the strip 710 may be disposable. Advantages of disposable strips 710 include elimination of problems associated with bacteria on a dropper end of the standard eye drop bottle 702. In other examples, the strip 710 may be reusable and washable.

FIG. 8 shows an eye drop system 800 and method according to an example of the invention. In FIG. 8, a standard eye drop bottle 802 is inserted into a holder 810. The holder 810 includes an upper arm 812, and a base 814. The standard eye drop bottle 802 is held within the upper arm 812. In one example, the upper arm 812 is adapted to hold different sizes of eye drop bottle 802. One example of a configuration within the scope of the invention includes a tapered hole that will hold narrow eye drop bottles 802 as well as wide eye drop bottles 802. Another example of a configuration within the scope of the invention includes a fork with widening arms. Other configurations are also within the scope of the invention.

A strip 810 as described in any of the previous examples is placed on the base 814 near a tip 804 of the eye drop bottle 802. In the example shown, the strip 810 includes a region 806 similar to the dissolvable portion 156 156 shown in FIG. 1B. In other examples, the region 806 may include geometry designed to modify adhesion of a drop of medication. In another example, the region 806 may include a hydrophilic portion, or a hydrophobic portion. In one example, the region 806 may include a different material than the remaining strip 810. The strip may be in a container as part of a continuous roll as previously described. The container holding the roll of strips could be made to dock with the base 814 so that the strip is well-positioned and stable to receive a partial drop from the eye drop bottle.

In one example, the region 806 contains a well of hydrophobic material that is imprinted on the strip, and calculated to hold a volume of a fraction of a typical 50 microliter eye drop. Examples may include, but are not limited to, ½ drop, ⅓ drop, etc. In one example, the hydrophobic well includes fibrils that hold the drop in place with surface tension, while at the same time, the hydrophobic nature of the material in the well 806 keeps the drop from significantly absorbing into the strip.

In use, the eye drop bottle 802 is gently squeezed to release a small drop of eye medication. Due to the selected characteristics of the region 806 (geometry, material choice, hydrophobic/hydrophilic, etc.) a smaller amount of eye drop medication may be applied to the region 806.

As discussed above, in normal eye drops, an excessive amount of medicine is dispensed, with the understanding that only a small amount of the drop need actually come into contact with the eye. This can be wasteful of frequently expensive medication. Using the eye drop system 800, and strips as described in the present disclosure, a patient will be able to use only a medically necessary amount of medication, and extend the use of a standard eye drop bottle 802. In examples as described, the region 806 of the strip 810 may be self-limiting, and designed to only hold a medically necessary amount of medication.

To better illustrate the method and apparatuses disclosed herein, a non-limiting list of embodiments is provided here:

Example 1 includes a dispenser strip for application of eye medication. The dispenser strip includes a dissolvable carrier strip, and an amount of eye medication contained within the dissolvable carrier strip.

Example 2 includes the dispenser strip of example 1, wherein the dispenser strip is included in a roll of dispenser strip material.

Example 3 includes a dispenser strip for application of eye medication. The dispenser strip includes a carrier strip, and an amount of eye medication absorbed at least partially within the carrier strip.

Example 4 includes the dispenser strip of example 3, wherein the amount of eye medication is only absorbed on one end of the carrier strip.

Example 5 includes the dispenser strip of any one of examples 3-4, wherein the carrier strip includes a chemical that changes color with application of a chosen liquid medication to indicate an amount of absorbed medication.

Example 6 includes a strip for application of eye medication. The strip includes a carrier strip, and a dissolvable region on an end of the carrier strip, wherein the dissolvable end is sized and shaped to hold only a medically necessary amount of eye medication.

Example 7 includes the dispenser strip of example 6, wherein the dissolvable region is laminated onto the carrier strip.

Example 8 includes a method of applying medication to an eye. The method includes applying an amount of an eye medication to a first end of a carrier strip, at least partially absorbing the eye medication into the first end of the carrier strip, contacting an eye with the first end of the carrier strip, and transferring at least some of the eye medication from the first end of the carrier strip to the eye.

Example 9 includes the method of example 8, further including visualizing an amount of absorbed eye medication due to a color change in the carrier strip as the eye medication is absorbed.

Example 10 includes a method of applying medication to an eye. The method includes contacting an eye with an end of a dissolvable carrier strip, the dissolvable carrier strip having an amount of eye medication contained within the carrier strip, dissolving at least a portion of the dissolvable carrier strip when in contact with the eye, and transferring an amount of the eye medication to the eyes.

Example 11 includes a method of applying medication to an eye. The method includes applying a liquid drop of an eye medication to hydrophobic material region at an end of a strip, holding the liquid drop in place with surface tension that is enhanced by fibrils, contacting an eye with the strip, and transferring at least some of the liquid drop from the strip to the eye.

These and other examples and features of the present infusion devices, and related methods will be set forth in part in the above detailed description. This overview is intended to provide non-limiting examples of the present subject matter—it is not intended to provide an exclusive or exhaustive explanation.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-7. (canceled)

8. A method of applying medication to an eye, comprising:
applying an amount of an eye medication to a first end of a carrier strip;
at least partially absorbing the eye medication into the first end of the carrier strip;
contacting the eye with the first end of the carrier strip; and
transferring at least some of the eye medication from the first end of the carrier strip to the eye.

9. The method of claim 8; further including visualizing an amount of absorbed eye medication due to a color change in the carrier strip as the eye medication is absorbed.

10. A method of applying medication to an eye, comprising:
contacting an eye with an end of a dissolvable carrier strip, the dissolvable carrier strip having an amount of eye medication contained within the carrier strip;
dissolving at least a portion of the dissolvable carrier strip when in contact with the eye; and
transferring at least a portion of the amount of the eye medication to the eye.

11. A method of applying medication to an eye, comprising:
applying a liquid drop of an eye medication to a hydrophobic material region at an end of a strip;
holding the liquid drop in place with surface tension that is enhanced by fibrils;
contacting the eye with the strip; and
transferring at least some of the liquid drop from the strip to the eye.

12. A strip for application of eye medication, comprising:
a carrier strip; and
an amount of eye medication.

13. The strip of claim 12, wherein the carrier strip is a dissolvable carrier strip, and the amount of eye medication is contained within the dissolvable carrier strip.

14. The strip of claim 13, wherein the dissolvable carrier strip is included in a roll of dispenser strip material.

15. The strip of claim 12, wherein the amount of eye medication is absorbed at least partially within the carrier strip.

16. The strip of claim 15, wherein the amount of eye medication is only absorbed on one end of the carrier strip.

17. The strip of claim 15, wherein the carrier strip includes a chemical that changes color with application of a chosen liquid medication to indicate an amount of absorbed medication.

18. The strip of claim 12, wherein the carrier strip includes a dissolvable region on an end of the carrier strip, wherein the dissolvable end is sized and shaped to hold only a medically necessary amount of eye medication.
